(19) United States
(12) Patent Application Publication
(10) Pub. No.: US 2023/0317223 A1
(43) Pub. Date: Oct. 5, 2023

(54) SYSTEM AND METHOD OF INTEGRATION OF SERVICE ENGINE PLATFORM WITH AN EMR SYSTEM

(71) Applicant: Teledact Inc., Thornhill (CA)

(72) Inventor: Tommy Tsz Him CHEUNG, Thornhill (CA)

(21) Appl. No.: 18/192,598

(22) Filed: Mar. 29, 2023

Related U.S. Application Data
(60) Provisional application No. 63/324,660, filed on Mar. 29, 2022.
(60) Provisional application No. 63/428,033, filed on Nov. 25, 2022.

(51) Int. Cl. G16H 10/60; G16H 40/40

(52) U.S. Cl. CPC G16H 10/60; G16H 40/40

(57) ABSTRACT

A system and method for integrating a service engine platform with an EMR system. The service engine platform provides connectivity and programmability across one or more EMR system and provides communication across multiple communication channels including SMS, email, phone, text-to-voice and voice-to-text service. The Service Engine platform allows for full customization of its algorithms and analytics, integration with EMR and patient care workflow, driving better patient health outcomes and fulfilling physician care needs.

### CROSS REFERENCE TO RELATED APPLICATIONS

The application claims priority to and the benefit of US Utility Provisional Application Ser. No. 63/324,660, entitled “SYSTEM AND METHOD OF INTEGRATION OF SERVICE ENGINE PLATFORM WITH AN EMR SYSTEM”, filed on Mar. 29, 2022 and US Utility Provisional Application Ser. No. 63/428,033, entitled “SYSTEM AND METHOD OF INTEGRATION OF SERVICE ENGINE PLATFORM WITH AN EMR SYSTEM”, filed on Nov. 25, 2022, both of which are incorporated herein by reference in their entirety.

### BACKGROUND

The embodiments described herein relate to telemedicine, in particular, technologies related to integrations with EMR systems.

Electronic medical records (EMR) are used by doctors and clinician offices to store patient medical information including individual contact information, and patient medical treatment and history. Currently, some EMR systems, such as Oscar Pro EMR, do not have a built-in communication system, such as an instant messaging or text messaging system to communicate with patients. Further these systems also lack the ability to securely store messages in each patient's medical chart.

Patient care coordination and service navigation is complex and often involves a multitude of providers and organizations. There is a desire to provide a centralized application to assist patients and providers in care delivery will improve patient experience and also provide better health outcomes.

### SUMMARY

A system and method for integrating a service engine platform with an EMR system. The service engine platform provides connectivity and programmability across one or more EMR system and provides communication across multiple communication channels including SMS, email, phone, text-to-voice and voice-to-text service. The Service Engine platform allows for full customization of its algorithms and analytics, integration with EMR and patient care workflow, driving better patient health outcomes and fulfilling physician care needs.

### BRIEF DESCRIPTION OF THE DRAWINGS

FIG. 1 is a diagram illustrating Service Engine.

FIG. 2 is a systems diagram illustrating components of Service Engine system.

FIG. 3 is a process diagram illustrating a workflow of public vs insurance service coverage.

FIG. 4 is a process diagram illustrating a workflow of a health service referral selection.

FIGS. 5A to 5E are process diagrams illustrating an in-person visit workflow for the Service Engine platform.

FIGS. 6A to 6L are process diagrams illustrating a partner web appointment booking workflow for the Service Engine platform.

### DETAILED DESCRIPTION

Practical software application is essential for patients seeking both virtual and in-person physician care and all services available from the associated health care providers. In particular, integration with existing electronic medical records (EMR) system can improve in patient care and workflow. More information on EMR integration can be found in U.S. Provisional Patent Application Ser. No. 63/232,424, entitled “SYSTEM AND METHOD OF INTEGRATING TEXT MESSAGING READ NOTIFICATION WITH AN EMR SYSTEM”, filed on Aug. 12, 2021 and US Provisional Patent Application Ser. No. 63/23,247, entitled “SYSTEM AND METHOD OF INTEGRATING TEXT MESSAGING NOTIFICATION FEATURES WITH AN EMR SYSTEM”, filed on Aug. 12, 2021, the disclosures of which are incorporated herein by reference in their entirety.

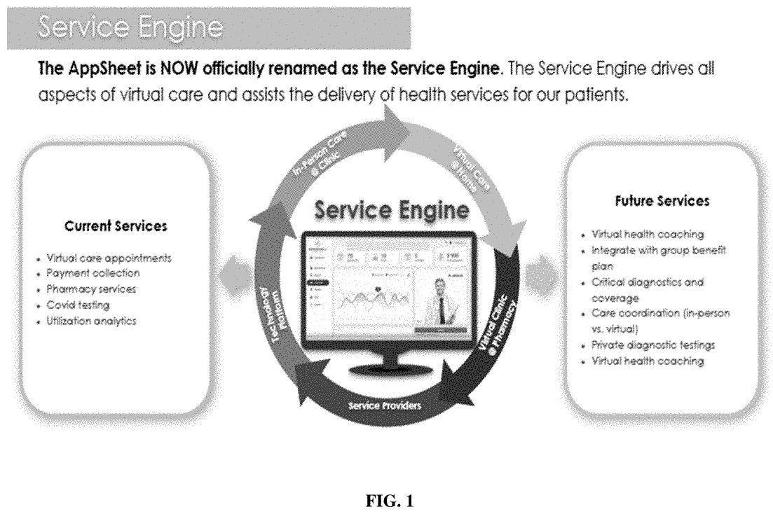

FIG. 1 is a diagram illustrating Service Engine. According to FIG. 1, a Service Engine platform is fully integrated with EMR and the patient care process. Based on predefined algorithms and data analytics. Patients and providers will receive prompts, notifications and guidance via various channels and systems that they interact with—EMR for physicians, mobile devices and SMS for patients and other communication methods.

According to FIG. 1, the Service Engine platform may connect to multiple pharmacies, clinics, health facilities, employer groups and other service organizations across the country, servicing thousands of patients each month. Algorithms have been set up to ensure service level targets and trigger necessary actions for physicians and other healthcare providers while they are servicing the patients.

According to FIG. 1, the Service Engine platform drives all aspects of virtual care and assists in the delivery of health services for patients. Service engine is a technology platform and connects to service providers. Furthermore, Service Engine provides services for in-person care at the clinics, virtual care at home and virtual clinic support at pharmacies.

According to FIG. 1, current services provided by Service Engine include:
Physician virtual and in-person online and phone appointment requests and tracking
Prescription fulfillment and pharmacy service delivery
Covid testing data integration and result processing
Specialist care referrals
Uninsured and private service online payment collection
Laboratory and diagnostic testing request and documentation
Payment collection
Pharmacy services
COVID testing
Utilization analytics

Future iterations of Service Engine may be configured to support the following services:
Group Benefit Plans integration with major insurance service providers
Virtual health coaching with collaboration partners
EAP and chronic disease management coordination
Critical diagnostics and coverage
Care coordination (in person vs virtual)
Private diagnostic testing
Virtual health coaching

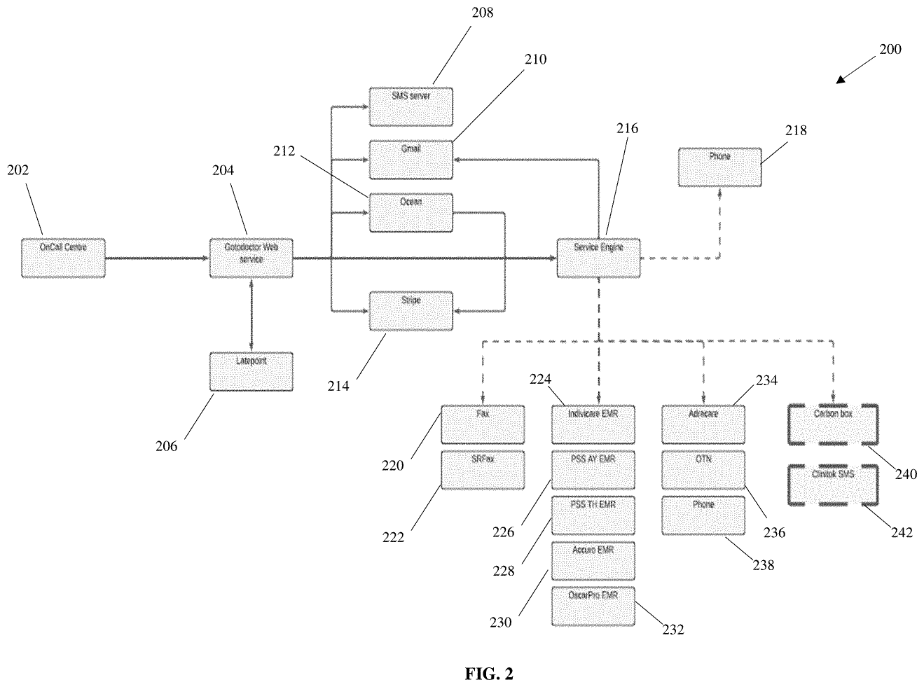

FIG. 2 is a systems diagram illustrating components of Service Engine system. According to FIG. 2, Service Engine system 200 consists of an initial input of virtual receptionists 202 (e.g., OnCall Centre). The information is sent to an online appointment system 204 (e.g., Gotodoctor Web Service) with an asynchronous connection to appointment scheduling applications 206 (e.g., Latepoint).

According to FIG. 2, the system splits into multiple connections including a SMS server 208, an email server 210 (e.g., Gmail), a connection to Ocean EMR system 212, a connection to Stripe payment system 214 and a connection to a Service Engine module 216. Service Engine module 216 will also provide connection to email server (e.g., Gmail), a mobile phone 218 (i.e., Android or Apple iOS) and a plurality of complimentary services and applications.

According to FIG. 2, complimentary services and application include connection to a faxing service 220 (e.g., SRFax 222), an electronic medical records system (e.g., Indivicare EMR 224, PSS AY EMR 226, PSS TH EMR 228, Accuro EMR 230, OscarPro EMR 232), a virtual care platform (e.g., Adracare 234 via Ontario Telemedicine Network 236 (OTN) or phone 238) and/or other messaging systems (e.g., Carbon Box 240 or Clinitok SMS 242).

According to further disclosures, the service engine platform provides connectivity and programmability across one or more EMR system and provides communication across multiple communication channels including short messaging service (SMS), email, phone, text-to-voice and voice-to-text services.

According to further disclosures, artificial intelligence (AI) and/or machine learning algorithms may be utilized to provide better analytics and dashboard information for presentation to the end user.

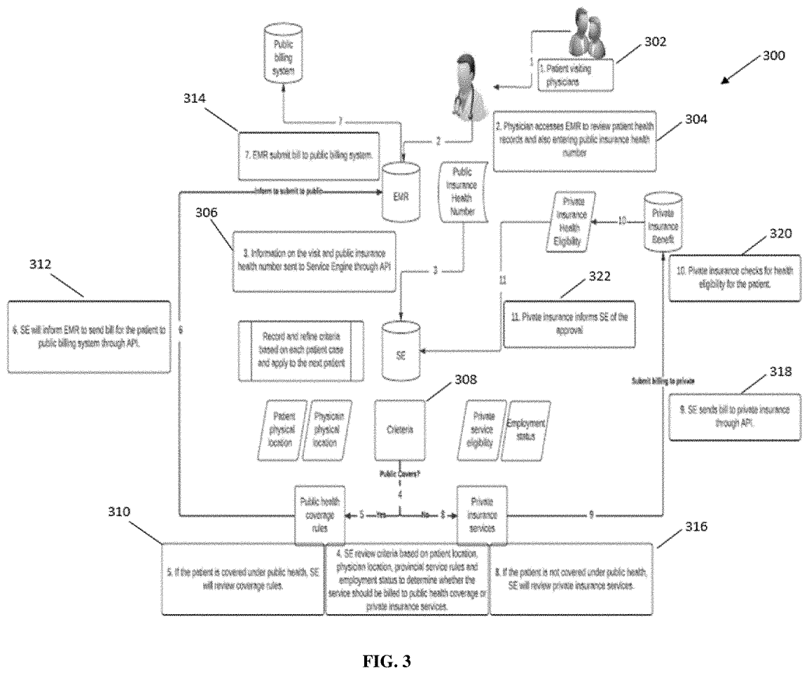

FIG. 3 is a process diagram illustrating a workflow of public vs insurance service coverage. According to FIG. 3, the workflow 300 initiates with a patient visiting a physician at step 302. The physician accesses an EMR to review the patient health records and also inputs public insurance health number at step 304. Next, the information on the visit and public insurance health number is sent to the Service Engine (SE) through an application program interface (API) at step 306.

According to FIG. 3, the service engine (SE) review criteria based on patient location, physician location, provincial service rules and employment status to determine whether the service should be billed to public health coverage or private insurance services at step 308. If the patient is covered under public health insurance, the service engine will review coverage rules at step 310. Next, the service engine will inform the EMR to send the bill for the patient to the public billing system through the API interface at step 312. Thereafter, the EMR will submit the bill to the public billing system at step 314.

According to FIG. 3, if the patient is not covered under the public health insurance, the service engine will review private insurance services at step 316. Next, the service engine sends the bill to the private insurance provide via the API interface at step 318. The private insurance checks for health eligibility for the patient at step 320. Finally, the private insurance informs SE of the approval at step 322.

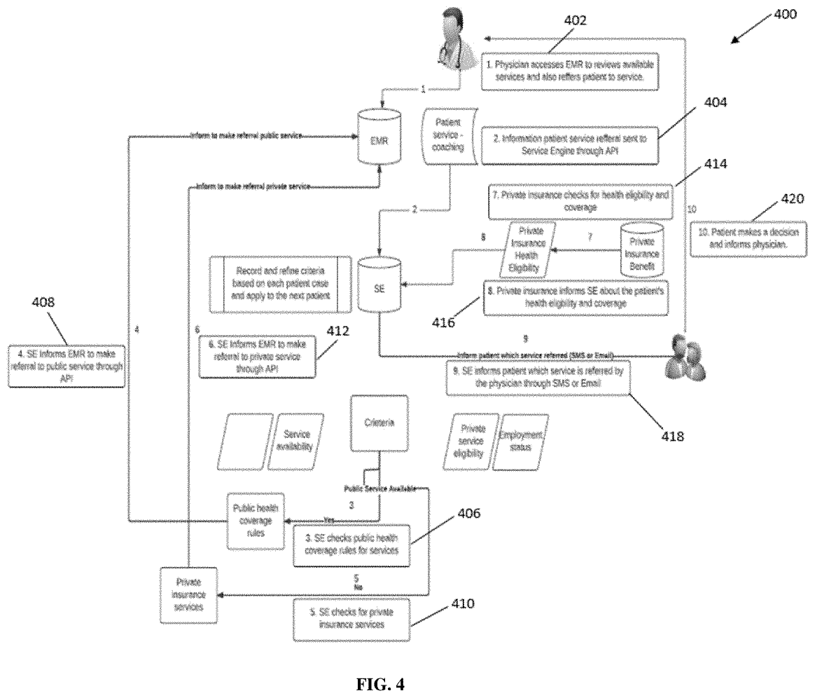

FIG. 4 is a process diagram illustrating a workflow of a health service referral selection. According to FIG. 4, workflow 400 initiates with a physician accessing EMR to reviews available services and also refers patient to service at step 402. The information patient service referral is sent to the service engine (SE) through an API interface at step 404. Next, the service engine (SE) checks public health coverage rules for services at step 406. The service engine (SE) informs the EMR to make a referral to public service through the API interface at step 408.

According to FIG. 4, the service engine checks for private insurance services at step 410. The service engine informs the EMR to make a referral to private service through the API interface at step 412. Thereafter, the private insurance checks for health eligibility and coverage at step 414. The private insurance informs the service engine (SE) about the patient's health eligibility and coverage at step 416. The service engine informs patient which service is referred by the physician through SMS or Email at step 418. Finally, the patient makes a decision and informs the physician at step 420.

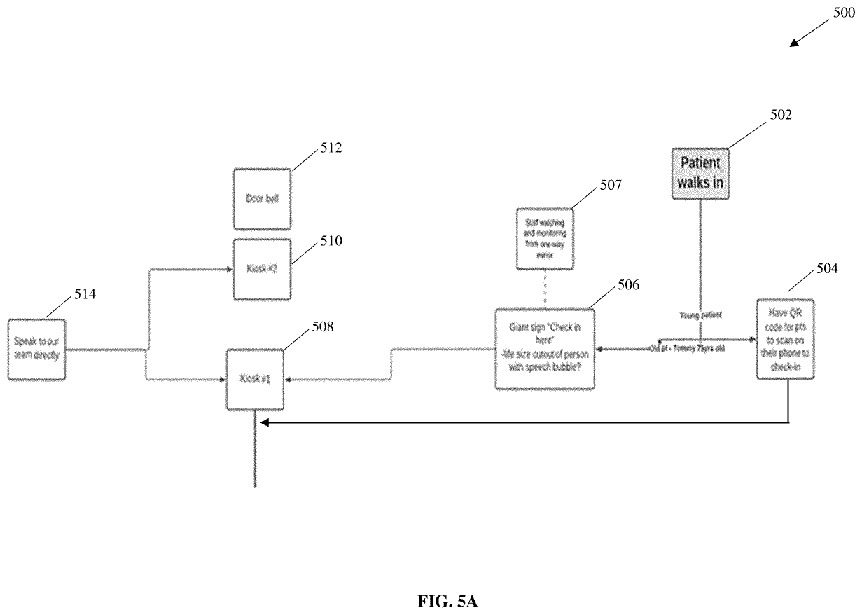

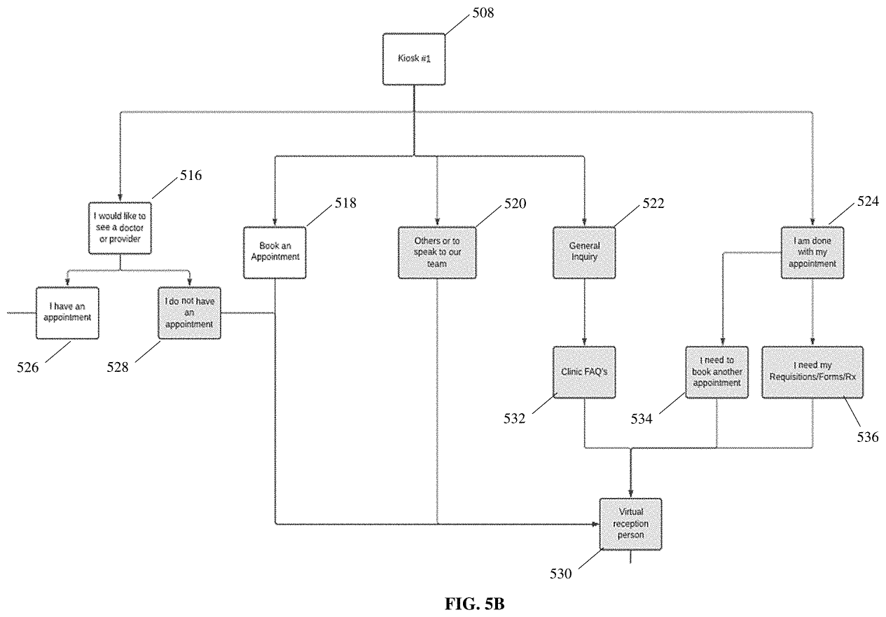

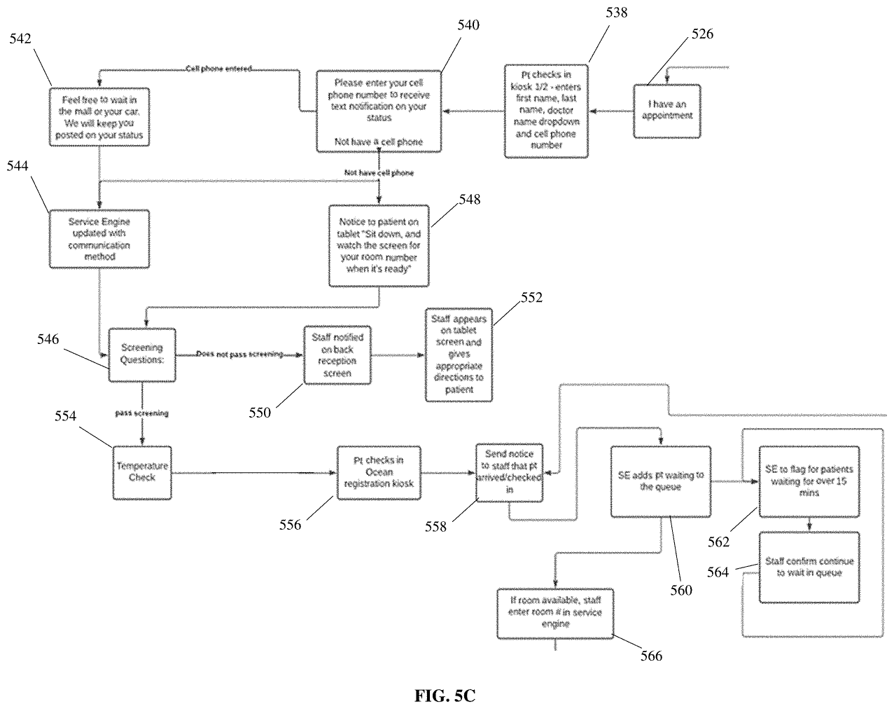

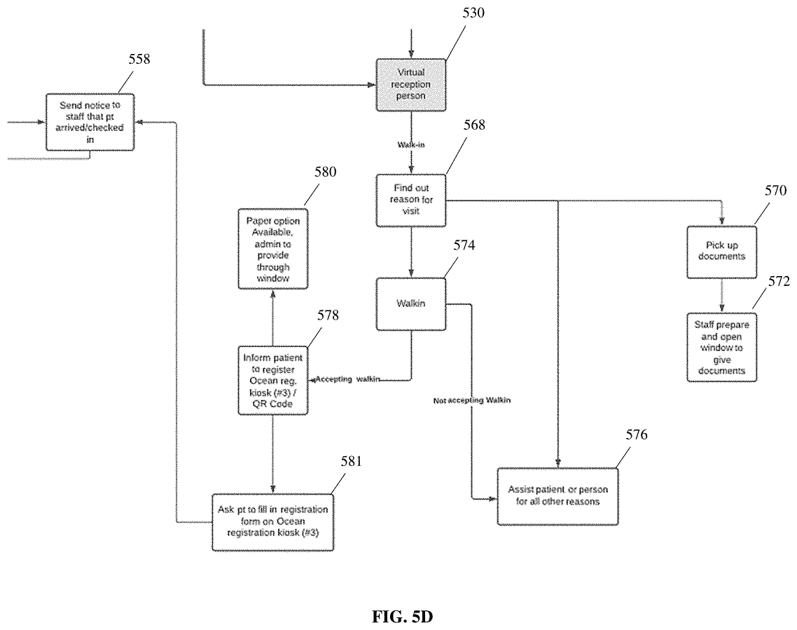

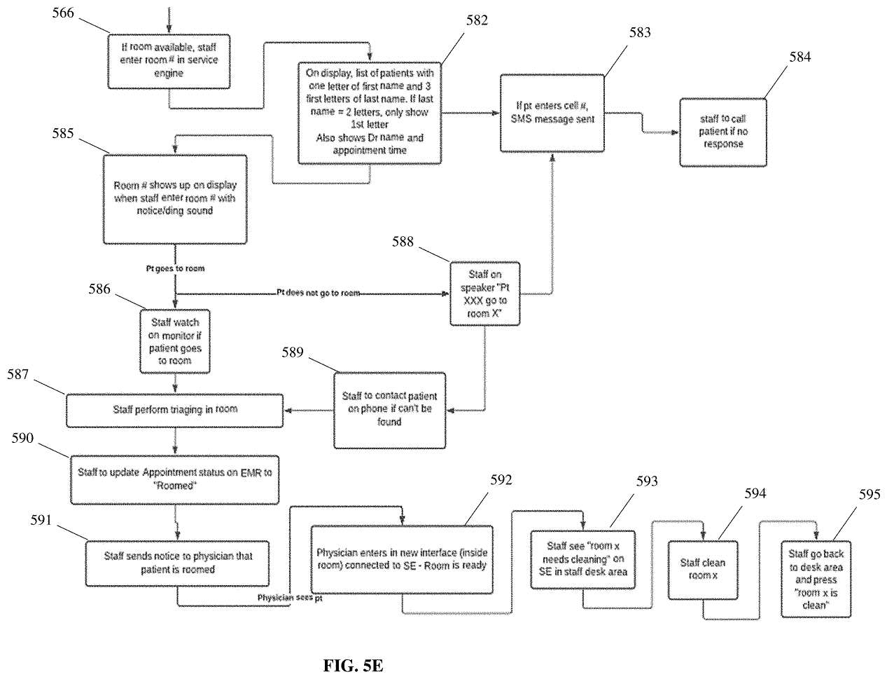

FIGS. 5A to 5E are process diagrams illustrating an in-person visit workflow for the Service Engine platform. FIG. 5A is the first diagram illustrating an in-person visit workflow and initiates when service engine platform 500 identifies a patient walks in at step 502. Service engine platform 500 determines whether the patient can initiate mobile check-in at step 504 (i.e., have QR code to scan on their phone to check-in) or physical check-in at step 506.

According to FIG. 5A, physical check-in at step 506 may include a large “Check in here” sign and/or a life size cutout of person with speech bubble. Furthermore, there may also be staff watching and monitoring from one-way mirror at step 507. The next step is to send the patient to a Kiosk for kiosk check-in (i.e., Kiosk1 508 or Kiosk2 510). Furthermore, the patient can speak to the team directly at step 514 by ringing a doorbell 512. Furthermore, upon completion of the mobile check-in at step 504, the process loops to the next step after Kiosk check-in at step 508 which continues further in FIG. 5B.

According to the disclosure, the workflow proceeds onto FIG. 5B continuing at Kiosk1 check-in at step 508 or mobile check-in at step 504. After going to Kiosk1 at step 508, the patient has a plurality of options, including seeing a doctor or provider at step 516, booking an appointment at step 518, speaking to others or the team at step 520, making a general inquiry at step 522 or making a decision that the patient is done with the appointment at step 524.

According to FIG. 5B, if the patient selects seeing a doctor or provider at step 516, the options thereafter including having an appointment at step 526 or do not have the appointment at step 528. If the patient selects to book an appointment at step 518 or selects to speak to others or the team at step 520, the next step is to direct to a virtual reception person at step 530.

According to FIG. 5B, if the patient selects general inquiry at step 522, the next step is directed to the clinic's FAQ at step 532. Thereafter, the process will direct to the virtual reception person at step 530.

According to FIG. 5B, if the patient selects that the patient is done with the appointment at step 524, the patient decides whether they need to book another appointment at step 534 or need to send in requisition, forms or prescriptions at step 536. Thereafter from step 534 and 536, the process than goes to the virtual reception person at step 530.

According to the disclosure, if the patient selects seeing a doctor or provider at step 516 and has an appointment at step 526, the process continues onto FIG. 5C. According to FIG. 5C, after having an appointment at step 526, the process moves onto step 538 which checks in kiosk 1 and 2 to enter patient info (e.g., first name, last name, doctor name dropdown and cell phone number).

According to FIG. 5C, the patient enters the cell phone number to receive text notification on status at step 540. If the cell phone number is entered at step 540, the process moves to step 542 where a notification message is sent. The notification message may include “Feel free to wait in the mall or your car. We will keep you posted on your status” at step 542. Thereafter, the Service Engine is updated with the communication method at step 544 and then moves onto the screening questions at step 546.

According to FIG. 5C, if the patient does not have a cell phone at step 540, a notice is sent to patient on a tablet at step 548. The notice may include the message “Sit down and watch the screen for your room number when it's ready” at step 548. After step 548, the process moves to screening questions at step 546.

According to FIG. 5C, at the screen questions step at 546, the process determines whether it passes screening. If it does not pass screening, the process moves to step 550 where the staff is notified on the back reception screen. Thereafter, the staff appears on tablet screen and gives appropriate directions to the patient at step 552.

According to FIG. 5C, if the process passes screening at step 546, the process moves to temperature check at step 554. Next, the patient and/or system checks into the Ocean registration kiosk at step 556. Next, the process moves to the step 558 where the staff appears on the tablet screen and gives appropriate directions to patient.

According to FIG. 5C, after step 558, the process moves to step 560 where the Service Engine adds the patient waiting to the queue. Thereafter, the Service Engine to flag for patients waiting for over 15 minutes at step 562 and the staff confirm continue to wait in queue at step 564.

According to FIG. 5C, after step 560 the process moves to step 566 where if the room is available, the staff enter room number in Service Engine.

According to FIG. 5D, the process continues on from virtual reception person at step 530 where the clinic finds out the reason for visit at step 568 where the patient picks up documents at step 570 and the staff prepares and open window to give documents at step 572.

According to FIG. 5D, at step 568 if the reason for visit is walk-in at step 574 and if the clinic is not accepting walk-in patients, the process moves to step 576 to assist patient or person for all other reasons at step 576. If the clinic is accepting walk-ins at step 574, the process moves to step 578 to inform patient to register Ocean registration, kiosk (#3) or QR Code.

According to FIG. 5D, at step 578, the process then moves to step 580 where there is a paper option available, admin to provide through window and a further process to ask the patient to fill in registration from on Ocean registration kiosk at step 581. The process then loops to step 558 to send notice to staff that patient has arrived or checked in.

According to the disclosure, the last step of FIG. 5C at step 566 where if the room is available, the staff enter room number in Service Engine continues onwards on FIG. 5E. The next step of the process is at step 584 where on display a list of patients with one letter of first name and 3 first letters of last name. If last name=2 letters, only show 1st letter. The process may also show the doctor name and appointment time.

According to FIG. 5E, after step 582, the process moves to step 583, if the patient enters a cell number, a short messaging service (SMS) message is sent. Thereafter, the staff is to call patient if no response at step 584. After step 582, the process may also move to step 585 where a room number shows up on display when staff enter room number with notice or notification (i.e., ding sound).

According to FIG. 5E, at step 585, if the patient goes to room, the process moves to step 586 where the staff watch on monitor if patient goes to room. Thereafter, the staff performs triaging in room at step 587.

According to step 585, if the patent does not go to room, the process moves to step 588 where there may be a staff notification where the staff on speaker may instruct patient to go to room (e.g., “patient 123 go to room X”). Thereafter, the staff will contact the patient on phone if can't be found at step 589 where the process than will loop to step 587 where the staff perform triaging in room.

According to FIG. 5E, after step 587 (staff triaging), the staff updates the appointment status on EMR to “Roomed” at step 590. Then the process moves to step 591 where the staff sends notice to physician that patient is roomed. Next, the physician sees the patient and moves to step 592 where the physician enters in new interface (inside room) connected to Service Engine—Room is ready.

According to FIG. 5E, after step 592, the staff see “room X needs cleaning” on Service Engine in staff desk area at step 593. The next step is for the staff to clean the room (i.e., room X) at step 594. Finally, the staff goes back to desk area confirms that the room is cleaned at step 595.

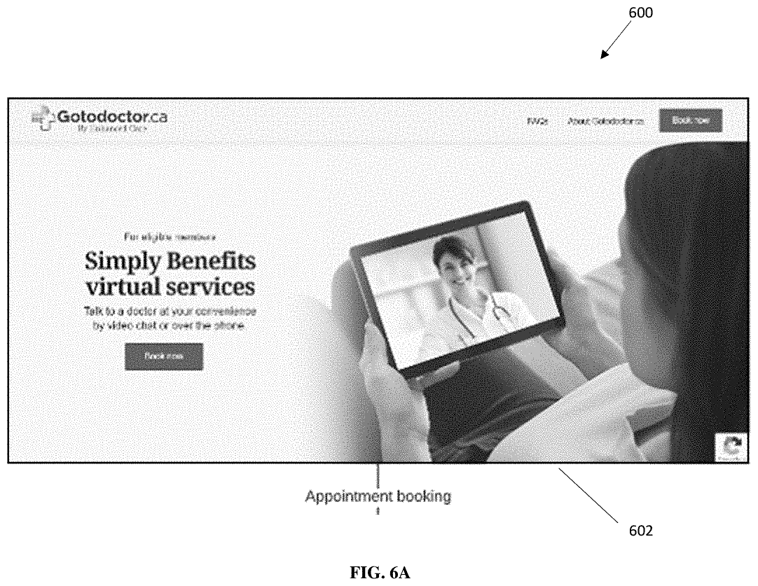

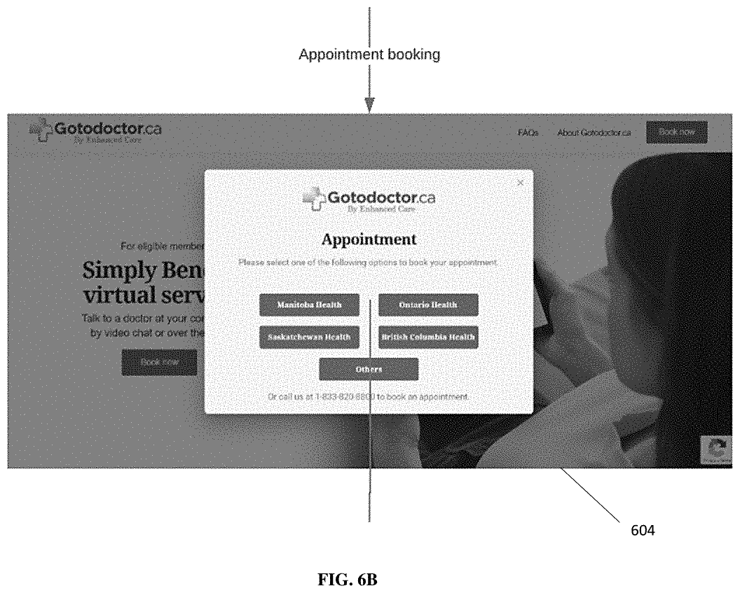

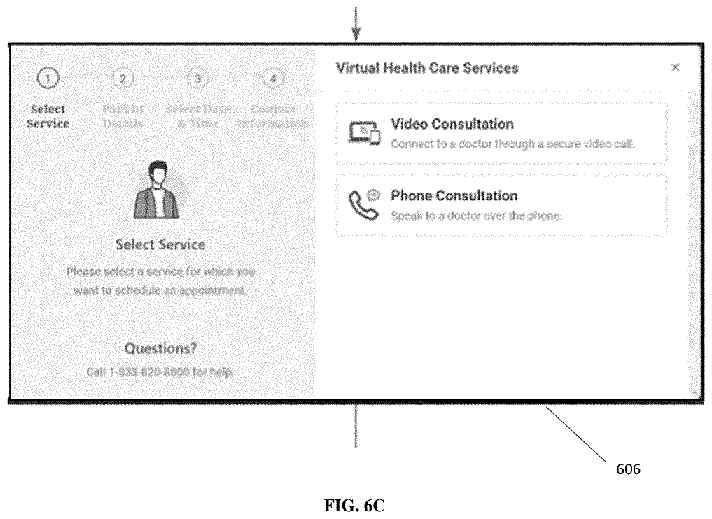

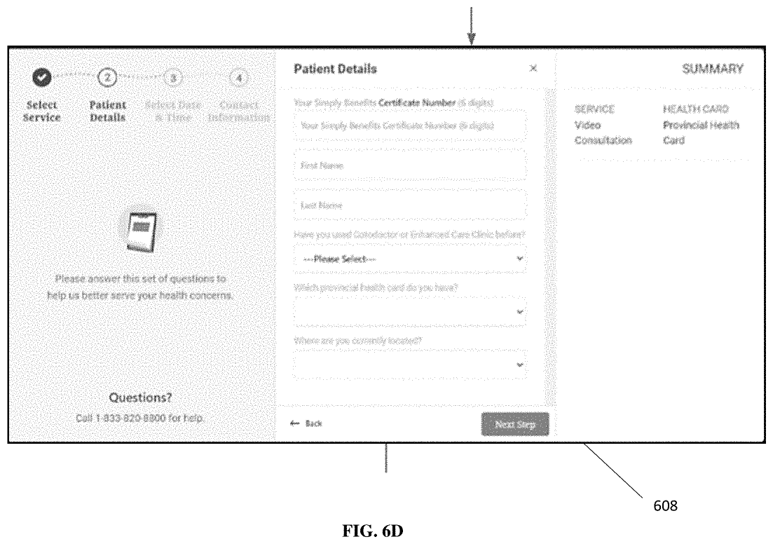

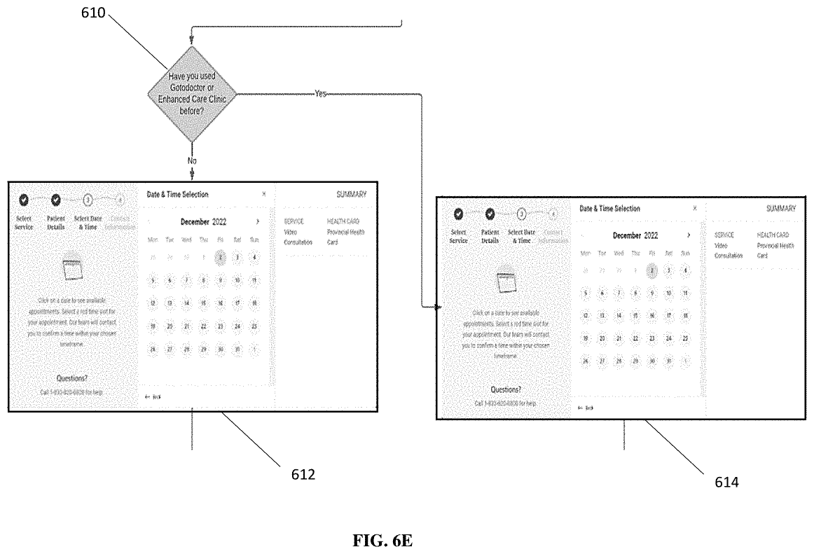

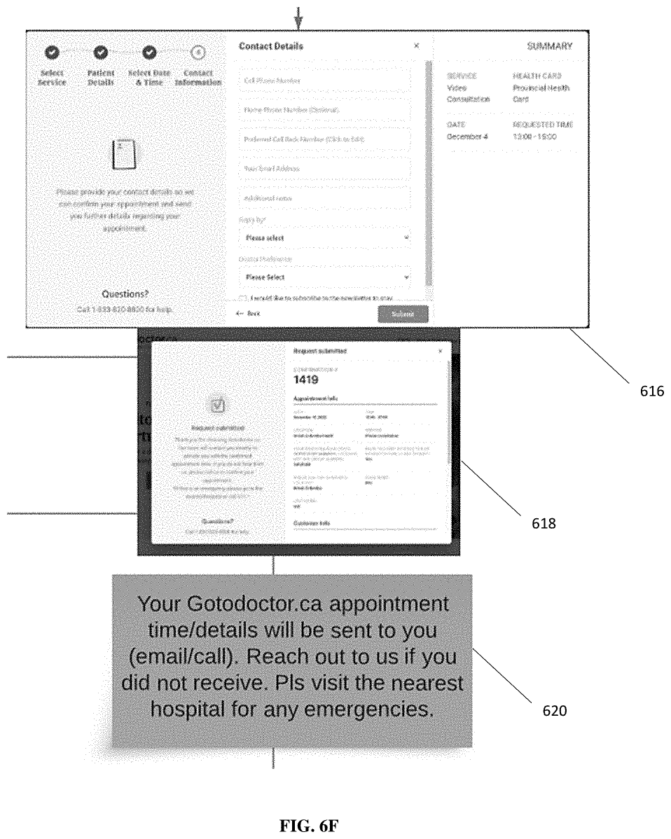

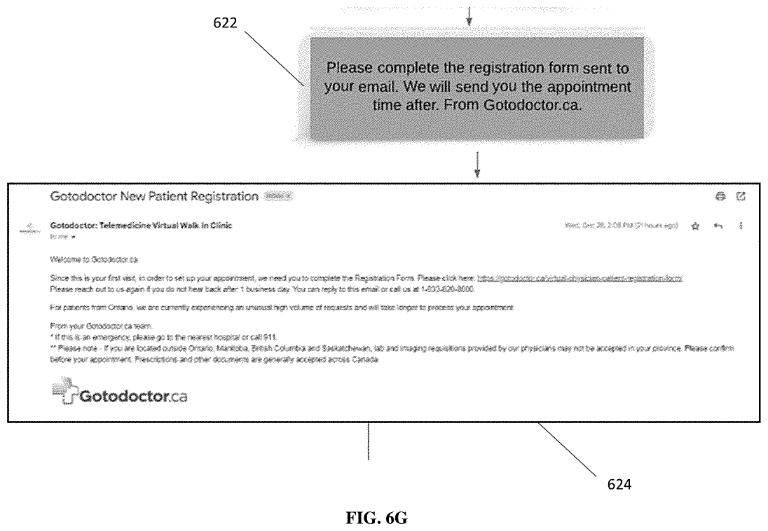

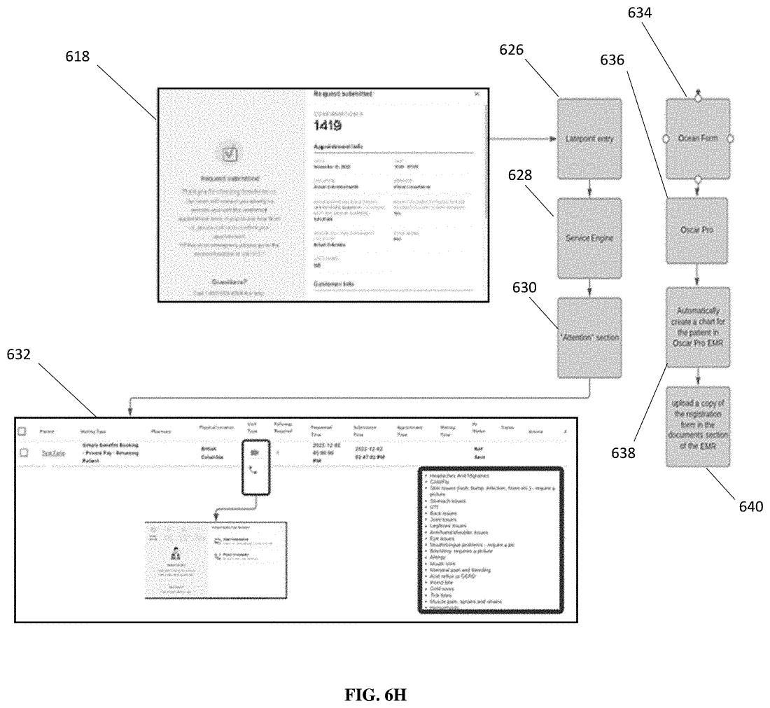

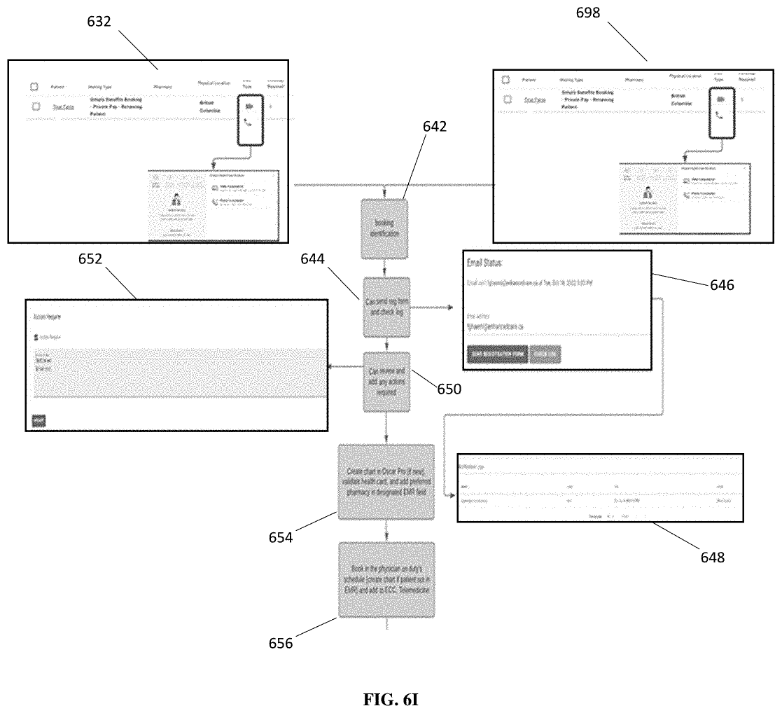

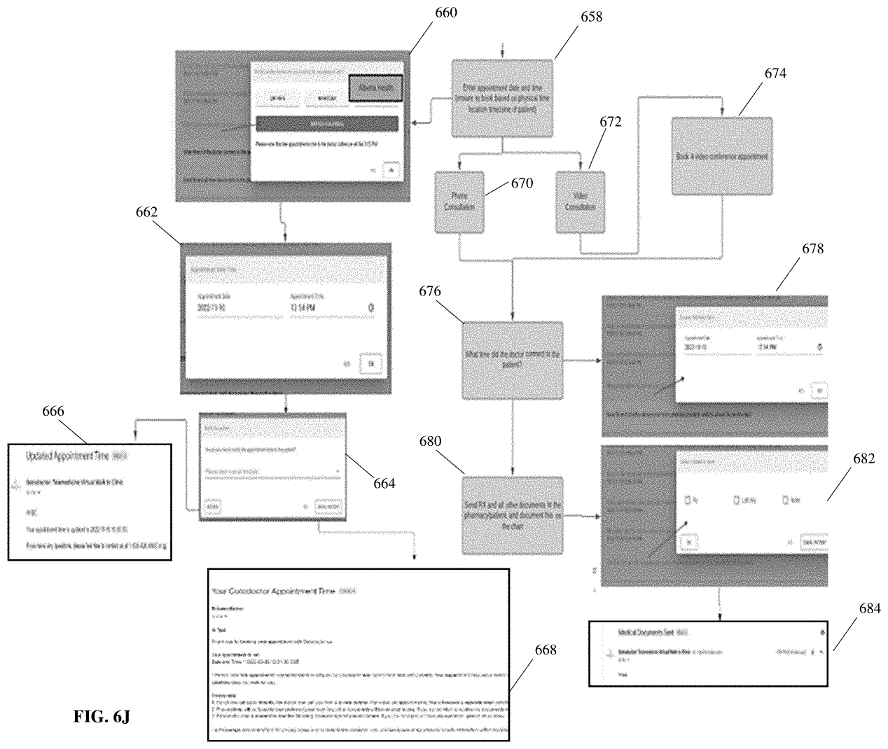

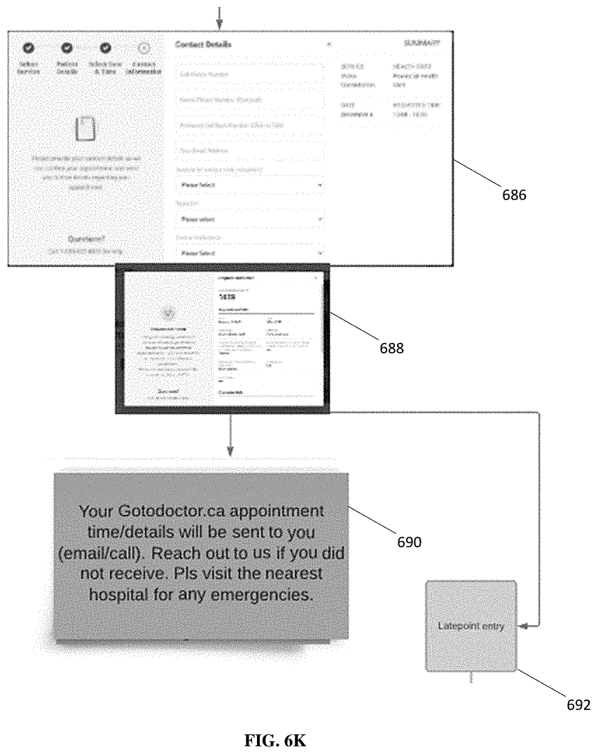

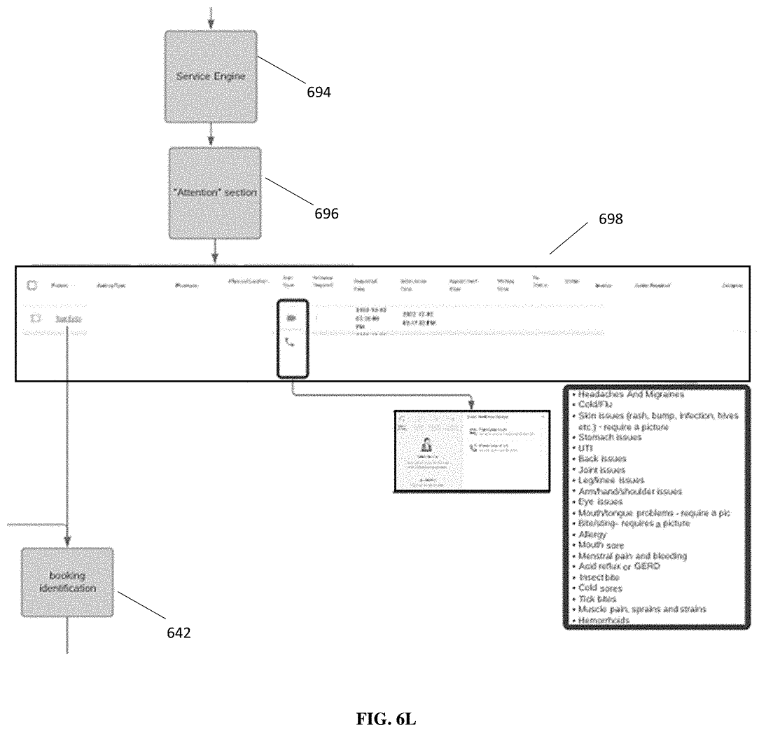

FIGS. 6A to 6L are process diagrams illustrating a partner web appointment booking workflow for the Service Engine platform. According to FIG. 6A, a partner web appointment booking workflow using the Service Engine platform 600 starts with visiting the partner web page (e.g., Gotodoctor.ca) at step 602 for an appointment booking.

According to the disclosure, this brings up the Appointment screen at step 604 which shows different portal options based on the province the patient resides in (e.g., Manitoba Health, Ontario Health, Saskatchewan Health, British Columbia Health, Others, etc.) as shown in FIG. 6B.

According to the disclosure, the next step is to select a Virtual Care Service at step 606. The Virtual Care Service can be a Video Consultation or a Phone Consultation as shown in FIG. 6C.

According to the disclosure, the next step is to provide Patient Details in step 608 as shown in FIG. 6D. Such info as Benefits Certificate Number, First Name, Last Name, Provincial Health Card number and location is provided. Once the patient enters all this data, they will input the data by clicking the “Next Step” button to proceed to the next screen.

According to the disclosure, after clicking the “Next Step” button, the system prompts the user whether they have used the portal site before (i.e., question such as “Have you used Gotodoctor or Enhanced Care Clinic before?”) at step 610 as shown in FIG. 6E. If the answer is No, the portal will proceed to the Select Date & Time screen at step 612. If the answer is Yes, the portal will proceed to the Select Date & Time screen at step 614.

According to the disclosure, the process proceeds to the next screen at FIG. 6F at step 616 where Contact Information is entered. Such fields as Cell Phone Number, Home Phone Number, Preferred Call Back Number, Email Address, Additional Notes, Reply By and Doctor Preference fields are completed. Once the fields are completed, the user will click on the “Submit” button.

According to FIG. 6F, upon clicking the “Submit” button, the Request is Submitted at step 618 where a Confirmation Number (i.e., 1419 and appointment details such as date, time, location, type of service, last name and certificate number is provided). Furthermore, a notification message may be provided at step 620. The notification message may include “Your Gotodoctor.ca appointment time/details will be sent to you (email/call). Reach out to us if you did not receive. Please visit the nearest hospital for any emergencies.”

According to the disclosure, the process proceeds to the next screen at FIG. 6G where the process prompts a further message of “Please complete the registration form sent to your email. We will send you the appointment time after. From Gotodoctor.ca” at step 622. Thereafter, an email provide next step instructions for New Patient registration is sent to the patient email address at step 624.

According to the disclosure, the process proceeds to the next screen at FIG. 6H where the process continues from step 618 where a Confirmation Number is provided. The next step is at step 626 for Latepoint entry. Thereafter the process is sent to the Service Engine module at step 628.

According to FIG. 6H, the next step is step 630 where the process moves to the “Attention” section. Thereafter, a portal screen is shown to provide info and data entry at step 632.

According to the disclosure, FIG. 6H also continues from step 624 from FIG. 6G wherein an Ocean form is provided at step 634. Thereafter, the process moves to Oscar Pro forms at step 636. Thereafter, the process automatically creates a chart for the patient in Oscar Pro EMR at step 638. Finally, the data is uploaded as a copy of the registration form in the documents section of the EMR at step 640.

According to the disclosure, the process proceeds to the next screen at FIG. 6I which proceed from step 632 and step 698 where the process moves to booking identification at step 642. The next step is to send registration form and check log at step 644 and provide email status at step 646 and notification logs at step 648.

According to FIG. 6I, the next step after step 644 is step 650 to review and add any actions required at step 650 and to provide an update action require screen at step 652.

According to FIG. 6I, the next step after step 650 is step 654 to create chart in Oscar Pro (if new), validate health card, and add preferred pharmacy in designated EMR field. Thereafter, the process moves to step 656 where the process books in the physician on duty's schedule (create chart if patient not in EMR) and add to ECC and Telemedicine.

According to the disclosure, the process proceeds to the next screen at FIG. 6J, after step 656, the process moves to step 658 where the patent enters appointment date and time (ensure to book based on physical time location time zone of patient). Thereafter, the patient selects the province/service provide and pressed OK to proceed, at step 660.

According to FIG. 6J, the next screen is for appointment date/time is provided at step 662. Thereafter, the next screen is to notify the patent at step 664 to provide an updated appointment time emails at step 666 and step 668.

According to FIG. 6J, returning back to step 658, after entering appoint date/time, the process branches into phone consultation at step 670 and video consultation at step 672. For video consultation at step 672, the next step is to book a video conference appointment at step 674. Thereafter, the system confirms the time that the doctor to connect to the patient at step 676 where the patient is prompted to provide appointment date and time at step 678.

According to FIG. 6J, after step 676, the next step is step 680 where the process sends the prescription and all other documents to the pharmacy and patient and document this on the chart. The next screen provides the patient the options to send a prescription (Rx), lab requisition and/or notes at step 682. Finally, an email notification is sent at step 684.

According to the disclosure, returning to FIG. 6E, if the choice is Yes at step 610 (i.e., Have you used Gotodoctor Enhanced Care Clinic before), the portal will proceed to the Select Date & Time screen at step 614 and then moves to FIG. 6K. According to the FIG. 6K step 686 where Contact Information is entered. Such fields as Cell Phone Number, Home Phone Number, Preferred Call Back Number, Email Address, Additional Notes, Reply By and Doctor Preference fields are completed. Once the fields are completed, the user will click on the “Submit” button.

According to FIG. 6K, upon clicking the “Submit” button, the Request is Submitted at step 688 where a Confirmation Number (i.e., 1419 and appointment details such as date, time, location, type of service, last name and certificate number is provided). Furthermore, a notification message may be provided at step 690. The notification message may include “Your Gotodoctor.ca appointment time/details will be sent to you (email/call). Reach out to us if you did not receive. Please visit the nearest hospital for any emergencies.”. According to FIG. 6K, step 688 also branches onto Latepoint entry at step 692.

According to the disclosure, the process proceeds onto the next step 694 at the Service Engine module in FIG. 6L. According to FIG. 6L, the next step is step 696 where the process moves to the “Attention” section. Thereafter, a portal screen is shown to provide info and data entry at step 698. Finally, the process looks to step 642 for booking identification which loops to FIG. 6I to continue with the process.

According to the disclosure, the service engine platform (including the service engine module) is configured to integrate with existing EMR systems and create data exchange protocol compliant with LIS1-A and LIS2-A. These are Clinical and Laboratory Standard Institute (CLSI) standards for electronic data exchange.

According to the disclosure, a full-function Data Exchange Layer (DXL) was implemented in the service engine application for insurance member eligibility and real-time data confirmation to facilitate service delivery for physicians. This secure data exchange protocol enables data exchange capability amongst legacy insurance systems, industry-standard 256-bit AES encryption front end user interface/control and data storage, and Fast Healthcare Interoperability Resource (FHIR)/Representation State Transfer (Rest) Application Programming Interface (API) based on Electronic Medical Record systems. Integration of the DXL layer enables further integration and data exchange with insurance systems (i.e., Blue Cross).

According to the disclosure, the service engine platform was configured to integrate with the FHIR API for the different EMR systems including OscarPro and Juno. Furthermore, the service engine platform was also integrated into other technological platforms, including LEMP, Laravel used in our front end user interface, FHIR API, Rest API in the OscarPro and Juno EMR, industry-standard 256-bit AES (Advanced Encryption Standard) Ocean & CognisantMD, legacy systems in the insurance and government payer resources, payment modules from Stripes and communication modules from Google works, SMS, email and phone systems.

According to the disclosure, the service engine platform can be further expanded to include the integration and data exchange for the following:
closed and secure systems like those with Third Party Administrator (TPA) for benefits and insurance claims processing
redefine overall data architecture and logic of databases to ensure data exchange with asymmetric and symmetric encryption while providing near real-time capability for various record
enhanced user control and access for personal health and personal data privacy requirements
investigate data connectivity possibility with other health informatic standards
Future health information exchange and connectivity using our DXL service engine application.
A proprietary communication and information exchange layer prototype in our service engine API schema connecting beyond EMR subsystems
An expansion of our system to retrieve patient registration, coverage, and service needs, combining with resource dataset and pre-define algorithm to support real-time decision making process for physicians and providers
Further integration of more than 1 insurance payers with the overall service engine architecture to accelerate patient care through our proprietary secure communication layer between different entities using SMS, email, phone, text-to-voice, and voice-to-text services
Proactive care recommendations based on patient and resources availability dataset
Cost-saving programs for insurance providers and their subscribers
Accelerated care navigation for specialist, diagnostic testing and home care to alleviate current long wait time and service backlog
Streamline virtual and in-person care by connecting and programming across EMR and clinical decision datasets
New health services to be integrated with our Service Engine application.

According to the disclosure, a service engine system for delivery of virtual care and health care service for patients is disclosed. The service engine system comprises a service engine module, a web portal, an appointment booking and reservation module, an email server configured to support one or more email systems, a short messaging service (SMS) server, a payment module configured to support credit card payments, a call center support module configured to support technical support, a fax server configured to support faxing services, a telephony module configured to support phone services and an electronics medical records (EMR) module configured to support EMR systems.

According to the disclosure the service engine module of the service engine system is configured to integrate the various services and modules to deliver virtual care and real-time health care services for the patient. The telephony module of the service engine system is configured to support land-line phones and cellular mobile phones.

According to the disclosure, the EMR module of the service engine system is configured to support OscarPro and Ocean EMR systems. The SMS server of the service engine system is configured to support the Swift SMS gateway and Clinitok SMS gateway.

According to the disclosure, the service engine system is configured to support third party middleware providers for health care procurement. The service engine system is configured to integrate with health service providers including Blue Cross and provincial and state health care providers. Furthermore, the service engine system is configured to provide services selected from a list consisting of virtual care appointments, payment collection, pharmacy services, covid testing, utilization analytics, virtual health coaching, insurance group benefits integration, critical diagnostics, care coordination, diagnostics testing.

According to the disclosure, a method of coordinating a walk-in, in-person patient, using a service engine system by a medical clinic staff is disclosed. The method comprising the steps of receiving the walk-in patient at a medical clinic, processing the patient as a mobile check-in or kiosk check-in, if kiosk check-in, receiving the patient at a kiosk at the clinic, determining whether patient would like to see a doctor or have other patient requests.

According to the disclosure, if the patient likes to see doctor and has an appointment, the method further comprising completing registration to check in at kiosk, updating the Service Engine system with cell phone communication method, screening the patient by requesting more patient data, conducting temperature check.

According to the disclosure, if other patient requests, the method further comprising, directing the patient to a virtual reception, determining the reason for the visit; retrieving relevant documents, determine whether the clinic is accepting walk-in patients. If not, assisting patient for other reasons, if accepting walk-in patients, informing patient to register with EMR system (Ocean).

According to the disclosure, the method further comprises the steps of sending notice to staff that patient has checked in, adding patient to Service Engine queue and monitor patient queue. If the if room if available, staff enters room data in Service Engine system and informing patient that room/slot available for appointment.

According to the disclosure, the method further comprises the steps of performing triage in room by the staff, updating the appointment status in EMR system to occupied, sending notice to physician to see patient. The physician enters new computer interface inside room connected to Service Engine to conduct patient appointment and upon completion of patient appointment, conduct a cleaning protocol by the staff.

According to the disclosure, if the patient conducts a mobile check-in, enable the patient to check-in using their mobile phone using a QR code. According to the disclosure, the other patient request of the method is selected from a list consisting of booking an appointment, speaking to team, general inquiry, reviewing clinic frequently asked questions (FAQ) and confirming appointment details.

According to the disclosure, the step of completing registration further comprising entering first name, last name, doctor name and cell phone contact. The step of screen patient further comprise asking screening questions.

According to the disclosure, the Service Engine system flags patients waiting over 15 minutes and prompts the staff to confirm that patient is in wait queue. Furthermore, the step of informing patient further comprising displaying room number on sign, sending SMS text, calling patient cell, sending email, announcing on speaker.

According to the disclosure, the cleaning protocol of the method further comprises the staff sees notice of “room needs cleaning” on Service Engine system, the staff cleans the room and the staff goes back to Service Engine system and updates the system that the room is cleaned.

According to the disclosure, a method of booking a web appointment for health services using a Service Engine system is disclosed. The booking appointment method comprises the steps of visiting a web portal web site to book the appointment, selecting a health care provider, selecting a video consultation service or a phone consultation service, inputting patient data, selecting date and time for the appointment, inputting contact data, submitting an appointment request, receiving a confirmation number, receiving a notification of the confirmation and appointment details and updating contact and appointment data at an electronics medical record (EMR) system.

According to the disclosure, the service engine system of the web appointment method is configured to integrate with one or more contact system, ERM system and messaging system to deliver real-time health care services and notifications to the patient. The health care provider of the web appointment method is selected form a list consisting of Blue Cross, Manitoba health, Ontario Health, Saskatchewan Health, British Columbia Heath or other service providers.

According to the disclosure, the patient data of the web appointment method is selected form list consisting of certificate number, first name, last name, health card number and location. The contact data of the web appointment method is selected form list consisting of cell phone number, home phone number, preferred contact number, email address, doctor preference and additional notes. The notification of the web appointment method is received by email, short messaging service (SMS) and phone text-to-voice notification.

Implementations disclosed herein provide systems, methods and apparatus for generating or augmenting training data sets for machine learning training. The functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor. A “module” can be considered as a processor executing computer-readable code.

A processor as described herein can be a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, or microcontroller, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, any of the signal processing algorithms described herein may be implemented in analog circuitry. In some embodiments, a processor can be a graphics processing unit (GPU). The parallel processing capabilities of GPUs can reduce the amount of time for training and using neural networks (and other machine learning models) compared to central processing units (CPUs). In some embodiments, a processor can be an ASIC including dedicated machine learning circuitry custom-build for one or both of model training and model inference.

The disclosed or illustrated tasks can be distributed across multiple processors or computing devices of a computer system, including computing devices that are geographically distributed. The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.” While the foregoing written description of the system enables one of ordinary skill to make and use what is considered presently to be the best mode thereof, those of ordinary skill will understand and appreciate the existence of variations, combinations, and equivalents of the specific embodiment, method, and examples herein. The system should therefore not be limited by the above described embodiment, method, and examples, but by all embodiments and methods within the scope and spirit of the system. Thus, the present disclosure is not intended to be limited to the implementations shown herein but is to be accorded the widest scope consistent with the principles and novel features disclosed herein.

What is claimed:

1. A service engine system for delivery of virtual care and health care service for patients comprising:
a service engine module;
a web portal;
an appointment booking and reservation module;
an email server configured to support one or more email systems;
a short messaging service (SMS) server;
a payment module configured to support credit card payments;
a call center support module configured to support technical support;
a fax server configured to support faxing services;
a telephony module configured to support phone services; and
an electronics medical records (EMR) module configured to support EMR systems;
wherein the service engine module is configured to integrate the various services and modules to deliver virtual care and real-time health care services for the patient.

2. The service engine system of claim 1 wherein the telephony module support land-line phones and cellular mobile phones.

3. The service engine system of claim 1 wherein the EMR module is configured to support OscarPro and Ocean EMR systems.

4. The service engine system of claim 1 wherein the SMS server configured to support the Swift SMS gateway and Clinitok SMS gateway.

5. The service engine system of claim 1 is configured to support third party middleware providers for health care procurement.

6. The service engine system of claim 1 is configured to integrate with health service providers including Blue Cross and provincial and state health care providers.

7. The service engine system of claim 1 is configured to provide services selected from a list consisting of virtual care appointments, payment collection, pharmacy services, covid testing, utilization analytics, virtual health coaching, insurance group benefits integration, critical diagnostics, care coordination, diagnostics testing.

8. A method of coordinating walk-in, in-person patient, using a service engine system by a medical clinic staff, the method comprising the steps of:
receiving the walk-in patient at the medical clinic;
processing the patient as a mobile check-in or kiosk check-in;
if the selection is a kiosk check-in, receiving the patient at a kiosk at the clinic;
determining whether patient would like to see a doctor or have other patient requests;
if the patient likes to see doctor and has an appointment, the method further comprising:
completing registration to check in at the kiosk;
updating Service Engine system with cell phone communication method;
screening the patient by requesting more patient data;
conducting a temperature check;
if the selection is other patient requests, the method further comprising:
directing to a virtual reception on a computer;
determining a reason for the visit;
retrieving relevant documents;
determining whether clinic is accepting walk-in patients;
if the clinic is not accepting walk-in patients, assisting the patient for other reasons;
if the clinic is accepting walk-in patients, inform patient to register with EMR system;
sending a notice to staff that patient has checked in;
adding the patient to Service Engine queue and monitoring patient queue;
if a room available, the staff enters room available in the Service Engine system;
informing patient that room is available for appointment;
performing triage on the patient in the room by the staff;
updating the appointment status in the EMR system to occupied;
sending notice to physician to see patient;
physician enters new computer interface inside room connected to Service Engine system to conduct patient appointment; and
upon completion of patient appointment, conduct a cleaning protocol by the staff.

9. The method of claim 8 wherein if the selection is mobile check-in, enabling the patient to check-in using their mobile phone using a QR code.

10. The method of claim 8 wherein the other patient request is selected from a list consisting of booking an appointment, speaking to team, general inquiry, reviewing clinic frequently asked questions (FAQ) and confirming appointment details.

11. The method of claim 8 the step of completing registration further comprising entering first name, last name, doctor name and cell phone contact.

12. The method of claim 8 wherein the step of screen patient further comprises asking screening questions.

13. The method of claim 8 wherein the Service Engine system flags patients waiting over 15 minutes and prompts the staff to confirm that patient is in wait queue.

14. The method of claim 8 wherein the step of informing patient further comprising displaying room number on sign, sending SMS text, calling patient cell, sending email, announcing on speaker.

15. The method of claim 8 wherein the cleaning protocol further comprises:
staff sees notice of “room needs cleaning” on Service Engine system;
staff cleaning room; and
staff goes back to Service Engine system and updates system that room is cleaned.

16. A method of booking a web appointment for health services using a Service Engine system, the method comprising:
visiting a web portal web site to book the appointment;
selecting a health care provider;
selecting a video consultation service or a phone consultation service;
inputting patient data;
selecting date and time for the appointment;
inputting contact data;
submitting an appointment request;
receiving a confirmation number;
receiving notification of the confirmation and appointment details; and
updating contact and appointment data at an electronics medical record (EMR) system;
wherein the service engine system is configured to integrate with one or more contact system, ERM system and messaging system to deliver real-time health care services and notifications to the patient.

17. The method of claim 16 wherein the health care provider is selected form a list consisting of Blue Cross, Manitoba health, Ontario Health, Saskatchewan Health, British Columbia Heath or other service providers.

18. The method of claim 16 wherein the patient data selected form list consisting of certificate number, first name, last name, health card number and location.

19. The method of claim 16 wherein the contact data is selected form list consisting of cell phone number, home phone number, preferred contact number, email address, doctor preference and additional notes.

20. The method of claim 16 wherein the notification is received by email, short messaging service (SMS) and phone text-to-voice notification.
